(19) United States
(12) Patent Application Publication
(10) Pub. No.: US 2024/0306952 A1
(43) Pub. Date: Sep. 19, 2024

(54) SYSTEM AND METHOD FOR MONITORING HEALTH PARAMETERS

(71) Applicant: KNOW LABS, INC., Seattle, WA (US)

(72) Inventor: JOHN CRONIN, Seattle, WA (US)

(21) Appl. No.: 18/608,455

(22) Filed: Mar. 18, 2024

Related U.S. Application Data
(60) Provisional application No. 63/490,644, filed on Mar. 16, 2023.

(51) Int. Cl. A61B 5/145; A61B 5/00; A61B 5/0205; A61B 5/0245; A61B 5/11

(52) U.S. Cl. CPC A61B 5/14532; A61B 5/0022; A61B 5/0245; A61B 5/1118; A61B 5/14546; A61B 5/681; A61B 5/7203; A61B 5/725; A61B 5/02055; A61B 2560/0242; A61B 2562/0219; A61B 2562/0271

(57) ABSTRACT

A system for monitoring a health parameter is disclosed. The system comprises a device having one or more transmit antennas configured to transmit Activated RF Range radio waves over a space below the skin surface of a user and one or more receive antennas configured to receive a responded portion of the transmitted Activated RF Range radio waves. A network base module is configured to transmit or receive Activated RF Range radio waves from the device base module over the cloud network. The network base module comprises a create transfer function module configured to obtain an output signal of the device base module and a known output ground truth data. The create transfer function module generates an artificial intelligence (AI) correction algorithm between the output signal of the device base module and the known output ground truth data, and an execute transfer function module configured to execute the AI correction algorithm.

### CROSS-REFERENCE TO RELATED APPLICATIONS

This application claims priority to U.S. Provisional Application No. 63/490,644, filed on Mar. 16, 2023, the disclosure of which is incorporated herein by reference in its entirety.

### FIELD OF THE DISCLOSURE

The present disclosure is generally related to systems and methods of monitoring health parameters and, more particularly, relates to a system and a method of monitoring real-time glucose levels using millimeter-range radio frequency signals.

### BACKGROUND

The subject matter discussed in the background section should not be assumed to be prior art merely due to its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Diabetes is a medical condition in which a person's blood glucose level, also known as blood sugar level, is persistently elevated. Diabetes can result in severe medical complications, including cardiovascular disease, kidney disease, stroke, foot ulcers, and eye damage if left untreated. Typically, diabetes is caused by either insufficient insulin production by the pancreas, referred to as “Type 1 diabetes,” or improper insulin response by the body's cells referred to as “Type 2 diabetes.” Further, monitoring a person's blood glucose level and administering insulin when a person's blood glucose level is too high to reach the desired level may be part of managing diabetes. Depending on many factors, such as the severity of diabetes and the individual's medical history, a person may need to measure their blood glucose level up to ten times per day. Each year, billions of dollars are spent on equipment and supplies for monitoring blood glucose levels.

Moreover, regular glucose monitoring is a crucial component of diabetes care. Further, measuring blood glucose is generally a procedure that involves giving a blood sample at a clinic or hospital. Home glucose monitoring is also possible using a variety of devices. The blood sample is obtained by pricking the skin using a tiny instrument. A glucose meter or glucometer is a tiny instrument that measures the sugar in the blood sample. The majority of glucose monitoring methods and devices require a blood sample.

Currently, available glucose monitoring devices also require a blood sample, usually by pricking a needle under the skin and then using a glucose meter to determine the glucose level of a patient.

Therefore, there is a need for an improved system and method to monitor glucose levels with enhanced accuracy and without requiring a blood sample from the patient.

### SUMMARY

A system for monitoring a health parameter. In an embodiment, the system for monitoring a health parameter includes a device communicatively linked to a cloud network, wherein the device includes: one or more transmit antennas configured to transmit Activated RF Range radio waves over a space below the skin surface of a user; and a device base module configured to process the Activated RF Range radio waves, wherein the device base module is in communication with one or more receive antennas configured to receive Activated RF Range radio waves, including a responded portion of the transmitted Activated RF Range radio waves. The system for monitoring health parameters further includes a network base module configured to transmit or receive Activated RF Range radio waves from the device base module over the cloud network, wherein the network base module comprises: a create transfer function module configured to obtain an output signal of the device base module and a known output ground truth data, wherein the create transfer function module is configured to generate correlation algorithm between the output signal of the device base module and the known output ground truth data; and an execute transfer function module configured to execute the correlation algorithm.

Another example of a system for monitoring a health parameter can include a health parameter monitoring system, including a monitoring device configured for communicative coupling to an analysis system. The monitoring device including one or more transmit antennas configured to transmit radio-frequency (RF) analyte detection signals into a user over the space below a skin surface and one or more receive antennas configured to detect RF analyte signals that result from the RF analyte detection signals transmitted into the user. The monitoring device including an analog-to-digital converter connected to the one or more receive antennas and receiving the RF analyte signals detected by the one or more receive antennas. The monitoring device including a substrate on which the analog-to-digital converter and the one or more transmit antennas and the one or more receive antennas are fabricated, the transmit antennas and receive antennas forming one or more antenna arrays. Also the monitoring device including a sensor comprising at least one of: a movement sensor, a body temperature sensor, a body position sensor, and an electrocardiogram sensor, that senses user data during transmission of the RF analyte detection signals by the one or more transmit antennas and during detection of the RF analyte signals by the one or more receive antennas.

In another example, a health parameter monitoring method, including detecting an analyte in a user by transmitting radio-frequency (RF) analyte detection signals into the user over the space below a skin surface from one or more transmit antennas and detecting, using one or more receive antennas, RF analyte signals that result from the RF analyte detection signals transmitted into the user. The method including converting the detected RF analyte signals from analog signals to digital signals using an analog-to-digital converter connected to the one or more receive antennas. The method including sensing parameter data of the user using one of a movement sensor, a body temperature sensor, or a body position sensor, during transmission of the RF analyte detection signals and during detection of the RF analyte signals. The method including storing the digital signals and parameter data in a memory connected to the analog-to-digital converter.

In another example, a system for monitoring a health parameter including a device having one or more transmit antennas configured to transmit Activated RF Range radio waves over a space below the skin surface of a user and one or more receive antennas configured to receive a responded portion of the transmitted Activated RF Range radio waves. A network base module is configured to transmit or receive Activated RF Range radio waves from the device base module over the cloud network. The network base module comprises a create transfer function module configured to obtain an output signal of the device base module and a known output ground truth data. The create transfer function module generates an artificial intelligence (AI) correction algorithm between the output signal of the device base module and the known output ground truth data, and an execute transfer function module configured to execute the AI correction algorithm.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. In some examples, one element may be designed as multiple elements, or those multiple elements may be designed as one element. In some examples, an element shown as an internal component of one element may be implemented as an external component in another and vice versa. Furthermore, elements may not be drawn to scale. Non-limiting and non-exhaustive descriptions are described concerning the following drawings. The components in the figures are not necessarily to scale, emphasis instead being placed upon illustrating principles.

FIG. 1 a block diagram of a system for monitoring a health parameter in a user, according to an embodiment;

FIG. 2 illustrates a posterior view of a hand of the user with an approximate location of a cephalic vein and a basilic vein overlaid/superimposed, according to an embodiment;

FIGS. 3A-3B illustrate a cross-sectional view of a wrist with ulna and radius bones and the basilic vein, according to an embodiment;

FIG. 4 illustrates a functional block diagram of the system utilizing Activated RF Range radio waves to monitor blood glucose level in the user, according to an embodiment;

FIG. 5 illustrates a circuitry layout of the system on a substrate, according to an embodiment;

FIG. 6 illustrates the circuitry layout of the system overlaid on the wrist of the user, according to an embodiment;

FIG. 7 illustrates a flowchart of a method for execution on a device base module, according to an embodiment;

FIGS. 8A-B illustrate a flowchart of a method to create a transfer function, according to an embodiment;

FIG. 9 illustrates a flowchart of a method performed by a network base module, according to an embodiment; and

FIG. 10 illustrates a flowchart of a method performed by an execute transfer function module, according to an embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items or meant to be limited to only the listed item or items. The words “module” shall mean any software program or any hardware apparatus or any combination of software and hardware apparatus that performs the functions stated.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred systems and methods are now described.

Embodiments of the present disclosure will be described more fully from now on regarding the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

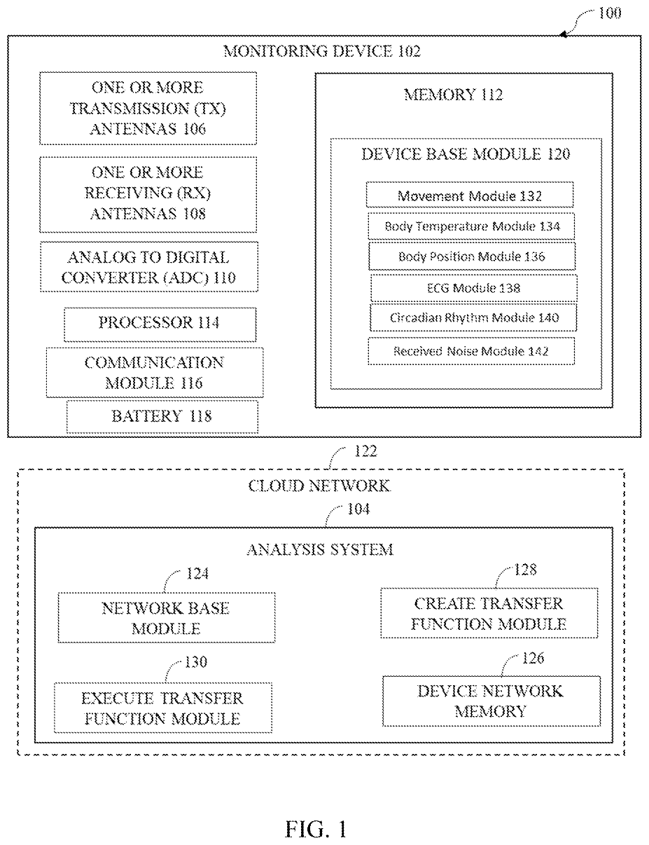

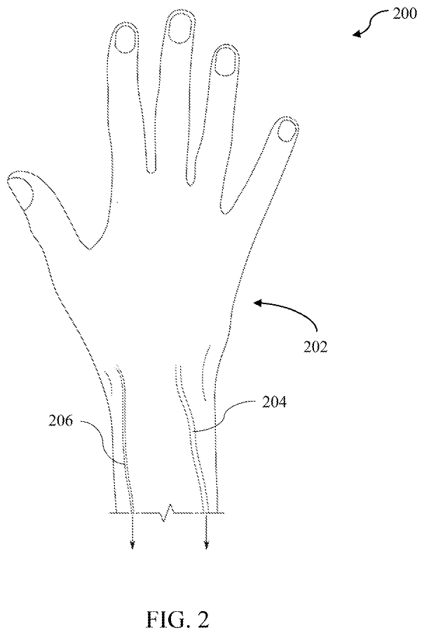

FIG. 1 illustrates a block diagram of a system 100 for monitoring the health parameters of a user, according to an embodiment. FIG. 1 is described in conjunction with FIGS. 2-10.

The system 100 may comprise a monitoring device 102 communicatively coupled to an analysis system 104. In one embodiment, the analysis system 104 may use a wireless and/or wired communication channel to connect to the monitoring device 102. The monitoring device 102 may be worn by the user. The monitoring device 102 may determine health parameters using radio frequency signals in the millimeter (mm) range. In one embodiment, the health parameters may include blood sugar or blood glucose levels. The system 100 may target specific blood vessels using the Activated RF Range radio frequency signals, with returning signals being detected, and the returning signals may correspond to the blood glucose level in the user.

In one embodiment, the system 100 may include integrated circuit (IC) devices (not shown) with transmit and/or receive antennas. Monitoring the blood glucose level in the specific blood vessels of the user using the Activated RF Range radio frequency signals involves the transmission of suitable Activated RF Range radio frequency signals below the user's skin surface. Corresponding to the transmission, a responded portion of the Activated RF Range radio frequency signals is received on multiple receive antennas. Further, the system 100 isolates and/or processes a signal from a particular location of the blood vessels in response to the received Activated RF Range radio frequency signals. The system 100 may output a signal from the received Activated RF Range radio frequency signals that correspond to the blood glucose level in the user. It can be noted that the monitoring device 102 may be worn by the user at various locations such as wrist, arm, leg, etc.

In one embodiment, the system 100 for monitoring the blood glucose level of the user uses radio waves in the “Activated RF Range” which is defined from 500 MHZ to 300 GHZ. The frequency range can be optimized to a shorter range depending upon the analysis algorithms used to analyze the received signals. The transmitted signals in the Activated RF Range are transmitted below the skin surface, and the received signals may be received with a delay period of the transmitted signals. This delay period can vary based upon the analysis algorithms used. An algorithm that requires a greater amount of data to get a higher correlation may result in a longer delay period to collect the additional data needed. In another embodiment, the received signals may have no delay or a standard delay. The received signals may be received on a single or a plurality of receive antennas, followed by isolating a signal from the Activated RF Range radio frequency signals at a particular location in response to the received Activated RF Range radio frequency signals, and outputting a signal that corresponds to the blood glucose level in the user in response to the isolated signal. In one embodiment, beamforming may be used in the receiving process to isolate the received signals from the transmitted Activated RF Range signals isolated from a specific location on a specific blood vessel to provide a high-quality signal corresponding to the blood glucose levels of the blood in the specific blood vessel. In another embodiment, Doppler effect processing may be used in the receiving process to isolate the received signals from the transmitted Activated RF Range signals isolated from a specific location on a specific blood vessel to provide a high-quality signal corresponding to the blood glucose levels in blood in the specific blood vessel In another embodiment isolation may not be needed where in a transfer function module evaluating all received signals can be correlated to blood glucose in blood in a specific blood vessel. It can be noted that analog and/or digital signal processing techniques may be used to implement beamforming and/or Doppler effect processing and digital signal processing of the received signals to dynamically adjust a received beam onto the desired location. In another embodiment, the beamforming and the Doppler effect processing may be used together to isolate the RF range radio signals received after the transmitted signals responded from the specific location in the specific blood vessel to provide the high-quality signal corresponding to the blood glucose levels in the blood in the specific blood vessel.

In one exemplary embodiment, Activated RF Range radio frequency signals of a higher frequency range of 122-126 gigahertz (GHz) providing a shallower penetration depth are used to monitor blood glucose levels. It can be noted that the shallower penetration depth may reduce undesirable received signals, wherein received signals may include interactions from bone and dense tissue such as tendons, ligaments, and muscle, which may reduce the signal processing burden and improve the quality of the desired signal that is generated from the location of the blood vessel. It can also be noted that bones are dielectric and semi-conductive. In addition, bones are anisotropic, so not only are bones conductive, they conduct differently depending on the direction of the flow of current through the bone. Alternatively, the bones are also piezoelectric materials. Therefore, radio frequency signals different from the range of 122-126 GHz in the shallower penetration depth may be required to monitor the blood glucose levels.

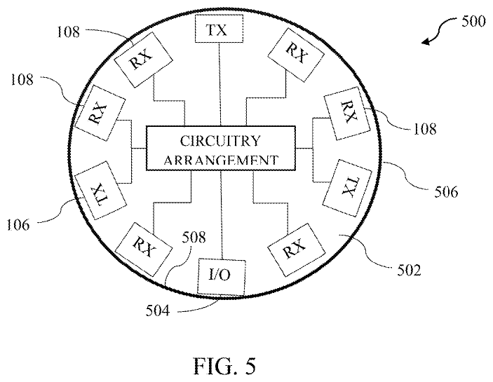

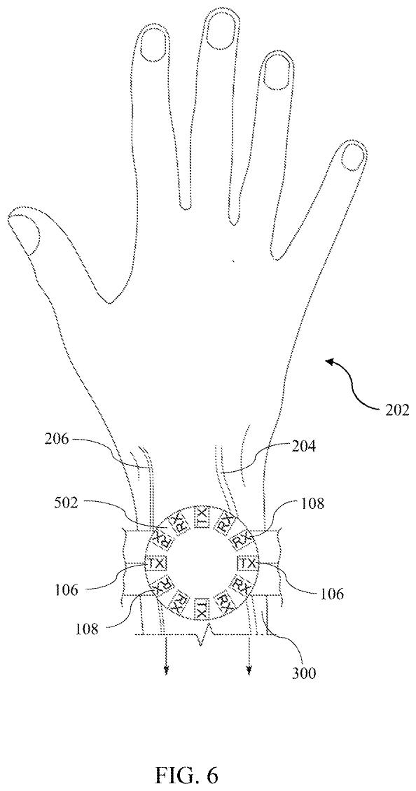

Further, the monitoring device 102 may comprise one or more transmission (TX) antennas 106, one or more receiving (RX) antennas 108, an analog to digital converter (ADC) 110, a memory 112, a processor 114, a communication module 116 and a battery 118. In one embodiment, the monitoring device 102 may be a wearable and portable device such as, but not limited to, a cell phone, a smartwatch, a tracker, a wearable monitor, a wristband, and a personal blood monitoring device. The one or more TX antennas 106 and the one or more RX antennas 108 may be fabricated on a substrate (not shown) within the monitoring device 102 in a suitable configuration. In one exemplary embodiment, at least two TX antennas and at least four RX antennas are fabricated on the substrate. The one or more TX antennas 106 and the one or more RX antennas 108 may correspond to a circuitry arrangement (not shown) on the substrate. The circuitry arrangement on the substrate is described in the later part of the detailed description in conjunction with FIGS. 5-6. Further, the ADC 110, the memory 112, the processor 114, the communication module 116, and the battery 118 may be fabricated on the substrate. Further, the communication module 116 may be configured to facilitate communication between the monitoring device 102 and the analysis system 104.

Further, the one or more TX antennas 106 and the one or more RX antennas 108 may be integrated into the circuitry arrangement. The one or more TX antennas 106 may be configured to transmit the Activated RF Range radio frequency signals at a pre-defined frequency. In one embodiment, the pre-defined frequency may correspond to a range suitable for the human body. For example, one or more TX antennas 106 transmit radio frequency signals at an Activated RF Range. Successively, the one or more RX antennas 108 may be configured to receive the radio waves as a result of the Activated RF Range being transmitted.

In one embodiment, the Activated RF Range radio frequency signals may be transmitted below the user's skin, and electromagnetic energy received by the receive antenna(s) may be a result responded from many parts such as fibrous tissue, muscle, tendons, bones, blood vessels, and the skin. It can be noted that effective monitoring of the blood glucose level is facilitated by an electrical response of blood molecules, such as pancreatic endocrine hormones, against the transmitted Activated RF Range radio frequency signals. It will be apparent to a skilled person that the pancreatic endocrine hormones such as insulin and glucagon are responsible for maintaining sugar or glucose level. Further, the electromagnetic energy received by the receive antennas are also from the blood molecules. Further, the ADC 110 may be coupled to the one or more RX antennas 108. The one or more RX antennas 108 may be configured to receive the responded Activated RF Range radio frequency signals as a result of the Activated RF Range applied to the transmission antennas 108. The ADC 110 may be configured to convert the responded Activated RF Range radio frequency signals from an analog signal into a digital processor readable format.

Further, the memory 112 may be configured to store the both the transmitted and received Activated RF Range radio frequencies. Further, the memory 112 may also store the converted digital processor readable format signals by the ADC 110. In one embodiment, the memory 112 may include suitable logic, circuitry, and/or interfaces that may be configured to store a machine code and/or a computer program with at least one code section executable by the processor 114. Examples of implementation of the memory 112 may include, but are not limited to, Random Access Memory (RAM), Read Only Memory (ROM), Hard Disk Drive (HDD), and/or a Secure Digital (SD) card.

Further, the system 100 may comprise a device base module 120 which may be fabricated within the memory 112 or otherwise be part of the monitoring device 102. The device base module 120 may be configured to store instructions for executing the computer program from the converted digital processor readable format of the ADC 110. The device base module 120 is configured to facilitate the operation of the processor 114, the memory 112, the one or more TX antennas 106, the one or more RX antennas 108, and the communication module 116. Further, the device base module 120 may be configured to create polling of the Activated RF Range radio frequency signals. It can be noted that the device base module 120 is configured to filter the Activated RF Range radio frequency signals received from the one or more RX antenna 108.

Further, the processor 114 may facilitate the operation of the monitoring device 102 with the analysis system 104 to perform functions according to the instructions stored in the memory 112. In one embodiment, the processor 114 may include suitable logic, circuitry, interfaces, and/or code that may be configured to execute a set of instructions stored in the memory 112. The processor 114 may be configured to run the instructions obtained by the device base module 120 to perform polling. The processor 114 may be further configured to collect real-time signals that are transmitted by the one or more TX antennas 106 and received by the one or more RX antennas 108 and may store the real-time signals in the memory 112. In one embodiment, the received real-time signals may be assigned as initial and updated radio frequency (RF) signals. Initial signals are the first readings of the received signals to start a comparison to other signals presented whereas updated radio frequency signals may be the current choices of the best RF signals to compare to the ground truth data. Examples of the processor 114 may be an X86-based processor, a Reduced Instruction Set Computing (RISC) processor, an Application-Specific Integrated Circuit (ASIC) processor, a Complex Instruction Set Computing (CISC) processor, and/or other processors. The processor 114 may be a multicore microcontroller specifically designed to carry-out multiple operations based upon pre-defined algorithm patterns to achieve the desired result.

Further, the processor 114 may take inputs from the monitoring device 102 and retain control by sending signals to different parts of the monitoring device 102. The processor 114 may access a Random Access Memory (RAM) that is used to store data and other results created when the processor 114 is at work. It can be noted that the data is stored temporarily for further processing, such as filtering, correlation, correction, and adjustment. Moreover, the processor 114 carries out special tasks as programs that are pre-stored in the Read Only Memory (ROM). It can be noted that the special tasks carried out by the processor 114 indicate and apply certain actions which trigger specific responses.

Further, the communication module 116 of the monitoring device 102 may communicate with the analysis system 104 via a cloud network 122. The communication module 116 may communicate with the analysis system 104 in any manner including, but not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). In one embodiment, various devices may be configured to have a communication module integrated over circuitry arrangement to connect with the analysis system 104 via various wired and wireless communication techniques, such as the cloud network 122. Examples of wired and wireless communication protocols that may be used include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols. In one embodiment, the battery 118 may be disposed on the substrate to power hardware modules of the monitoring device 102. The monitoring device 102 may be configured with a charging port to recharge the battery 118. It can be noted that the charging of the battery 118 may be performed by wired or wireless means.

The device base module 120 may include a movement module 132 that includes at least one sensor from the group of an accelerometer, a gyroscope, an inertial movement sensor, or other similar sensor. The movement module 132 may have its own processor or utilize the processor 114 to calculate movement of the user. Motion from the user will change the blood volume in a given portion of their body, and flow rate of blood in their circulatory system. This may cause noise, artifacts, or other errors in the real-time signals received by the RX antennas 108. The movement module 132 may compare the calculated motion to a motion threshold stored in memory 112. For example, the motion threshold could be movement of more than two centimeters in a one second period. The motion threshold could be near zero to ensure the user is stationary when measuring to ensure the least noise in the RF signal data. When calculated motion levels exceeds the motion threshold the motion module may flag the RF signals collected at the time stamp corresponding to the motion as potentially being inaccurate. In some embodiments, the motion module may compare RF signal data to motion data over time to improve the accuracy of the motion threshold. The movement module 132 may alert the user, such as with an audible beep or warning, or a text message or alert to a connected mobile device. The alert would signal to the user that they are moving too much to get an accurate measurement. The motion module may utilize the communication module 116 to communicate with the analysis system 104 in order to inform the create transfer function module 128 of the calculated motion of the user that corresponds with the received RF signal data. In this manner, the motion module may be simplified to just collect motion data and allow the create transfer function module 128 to determine if the amount of motion calculated exceeds a threshold that would indicate the received RF signal data is too noisy to be relied upon for a blood glucose measurement.

The device base module 120 may include a body temperature module 134 that includes at least one sensor from the group of a thermometer, a platinum resistance thermometer (PRT), a thermistor, a thermocouple, or other temperature sensor. The body temperature module 134 may have its own processor or utilize the processor 114 to calculate the temperature of the user or the user's environment. The user's body temperature, the environmental temperature, and the difference between the two will change the blood volume in a given part of their body, and flow rate of blood in their circulatory system. Variations in temperature from normal body temperature or room temperature may cause noise, artifacts, or other errors in the real-time signals received by the RX antennas 108. The body temperature module 134 may compare the measured temperature to a threshold temperature stored in memory 112. For example, the environmental temperature threshold may be set at zero degrees Celsius because low temperatures can cause a temporary narrowing of blood vessels which may increase the user's blood pressure. When the measured temperature exceeds the threshold the body temperature module 134 may flag the RF signals collected at the time stamp corresponding to the temperature as potentially being inaccurate. In some embodiments, the body temperature module 134 may compare RF signal data to temperature data over time to improve the accuracy of the temperature threshold. The body temperature module 134 may alert the user, such as with an audible beep or warning, or a text message or alert to a connected mobile device. The alert would signal to the user that their body temperature, or the environmental temperature is not conducive to getting an accurate measurement. The body temperature module 134 may utilize the communication module 116 to communicate with the analysis system 104 in order to inform the create transfer function module 128 of the measured user or environmental temperature that corresponds with the received RF signal data. In this manner, the body temperature module 134 may be simplified to just collect temperature data and allow the create transfer function module 128 to determine if the temperature measure exceeds a threshold that would indicate the received RF signal data is too noisy to be relied upon for a blood glucose measurement.

The device base module 120 may include a body position module 136 that includes at least one sensor from the group of an accelerometer, a gyroscope, an inertial movement sensor, or other similar sensor The body position module 136 may have its own processor or utilize the processor 114 to estimate the position of the user. The user's body position may change the blood volume in a given part of their body, and flow rate of blood in their circulatory system. This may cause noise, artifacts, or other errors in the real-time signals received by the RX antennas 108. The body position module 136 may compare the estimated position to a body position threshold stored in memory 112. For example, the monitoring device 102 may be on the user's wrist and the body position threshold may be based on the relative position of the user's hand to their heart. When a user's hand is lower than their heart, their blood pressure will increase, with this effect being more pronounced the longer the position is maintained. Conversely, the higher above a user's holds their arm above their heart, the blood pressure in their hand will be lower. The body position threshold may include some minimum amount of time the estimated body position occurs. When the estimated position exceeds the threshold the body position module 136 may flag the RF signals collected at the time stamp corresponding to the body position as potentially being inaccurate. In some embodiments, the body position module 136 may compare RF signal data to motion data over time to improve the accuracy of the body position threshold. The body position data may also be used to estimate variations is parameters such as blood pressure that correspond to the body position data so as to improve the accuracy of the measurements taken when the user in in that position. The body position module 136 may alert the user, such as with an audible beep or warning, or a text message or alert to a connected mobile device. The alert would signal to the user that their body position is not conducive to getting an accurate measurement. The body position module 136 may utilize the communication module 116 to communicate with the analysis system 104 in order to inform the create transfer function module 128 of the estimated body position data that corresponds with the received RF signal data. In this manner, the body temperature module 134 may be simplified to just collect temperature data and allow the create transfer function module 128 to determine if the body position exceeded a threshold that would indicate the received RF signal data is too noisy to be relied upon for a blood glucose measurement.

The device base module 120 may include an ECG module 138 that includes at least one electrocardiogram sensor. The ECG module 138 may have its own processor or utilize the processor 114 to record the electrical signals that correspond with the user's heartbeat. The user's heartbeat will impact blood flow. Measuring the ECG data may allow the received RF data to be associated with peak and minimum cardiac output so as to create a pulse wave form allowing for the estimation of blood volume at a given point in the wave of ECG data. Variations in blood volume may cause noise, artifacts, or other errors in the real-time signals received by the RX antennas 108. The ECG module 138 may compare the measured cardiac data to a threshold stored in memory 112. For example, the threshold may be a pulse above a 160 bpm, as the increased blood flow volume may cause too much noise in the received RF signal data to generate an accurate measure of blood glucose. When the ECG data exceeds the threshold the ECG module 138 may flag the RF signals collected at the time stamp corresponding to the ECG data as potentially being inaccurate. In some embodiments, the ECG module 138 may compare RF signal data to ECG data over time to improve the accuracy of the ECG data threshold or to improve the measurement of glucose at a given point in the cycle between peak and minimum cardiac output. The ECG module 138 may alert the user, such as with an audible beep or warning, or a text message or alert to a connected mobile device. The alert would signal to the user that their heart rate is not conducive to getting an accurate measurement or requires additional medical intervention. The ECG module 138 may utilize the communication module 116 to communicate with the analysis system 104 in order to inform the create transfer function module 128 of the measured ECG data that corresponds with the received RF signal data. In this manner, the ECG module 138 may be simplified to just collect ECG data and allow the create transfer function module 128 to determine if the ECG data exceeded a threshold that would indicate the received RF signal data is too noisy to be relied upon for a blood glucose measurement.

The device base module 120 may include a circadian rhythm module 140 (not shown) that includes at least one sensor measuring actigraphy, wrist temperature, light exposure, and heart rate. The circadian rhythm module 140 may have its own processor or utilize the processor 114 to calculate the user's circadian health. Blood pressure follows a circadian rhythm in that it increases on waking in the morning and decreases during sleeping at night. People with poor circadian health will often have higher blood pressure. These variations in blood pressure can noise, artifacts, or other errors or inaccuracies in the real-time signals received by the RX antennas 108. The circadian rhythm module 140 may compare the circadian data in such a way as to determine whether or not to a threshold is met. The threshold data may be stored in memory 112. This may be based on a single data point or many data points. For example, the threshold may be set as less than 6 hours of sleep in the last 24 hours. When the observed circadian health data exceeds the threshold the circadian rhythm module 140 may flag the RF signals collected at the time stamp corresponding to circadian health as potentially being inaccurate, or as needing an adjustment to account for the expected increase in the user's blood pressure. In some embodiments, the circadian rhythm module 140 may compare RF signal data to sleep data over time to improve the accuracy of the circadian rhythm thresholds. The circadian rhythm module 140 may alert the user, such as with an audible beep or warning, or a text message or alert to a connected mobile device. The alert would signal to the user that their recent sleep patterns are not conducive to getting an accurate measurement. The circadian rhythm module 140 may utilize the communication module 116 to communicate with the analysis system 104 in order to inform the create transfer function module 128 of the measured circadian data that corresponds with the received RF signal data. In this manner, the circadian rhythm module 140 may be simplified to just collect circadian rhythm data and allow the create transfer function module 128 to determine if the measure exceeded a threshold that would indicate the received RF signal data is too noisy to be relied upon for a blood glucose measurement, or if an alternative transfer function should be used to compensate for the detected circadian health.

The system 100 may include a received noise module 142 (not shown) that includes at least one sensor measuring background signals such as RF signals, Wi-Fi, and other electromagnetic signals that could interfere with the signals received by the RX antennas 108. The received noise module 142 may have its own processor or utilize the processor 114 to calculate the level of background noise being received. Background noise may interfere with or cause noise, artifacts, or other errors or inaccuracies in the real-time signals received by the RX antennas 108. The received noise module 142 may compare the level and type of background noise to a threshold stored in memory 112. The threshold may be in terms of field strength (volts per meter, and ampere per meter) or power density (watts per square meter). For example, the threshold may be RF radiation at greater than 300 μW/m2. When the background noise data exceeds the threshold the received noise module 142 may flag the RF signals collected at the time stamp corresponding to background noise levels as potentially being inaccurate. In some embodiments, the received noise module 142 may compare RF signal data to background noise over time to improve the accuracy of the noise thresholds. The received radiation module may alert the user, such as with an audible beep or warning, or a text message or alert to a connected mobile device. The alert would signal to the user that the current level of background noise is not conducive to getting an accurate measurement. The received noise module 142 may utilize the communication module 116 to communicate with the analysis system 104 in order to inform the create transfer function module 128 of the background noise data that corresponds with the received RF signal data. In this manner, the received noise module 142 may be simplified to just collect background noise data and allow the create transfer function module 128 to determine if the measure exceeded a threshold that would indicate the received RF signal data is too noisy to be relied upon for a blood glucose measurement, or if an alternative transfer function should be used to compensate for the detected circadian health.

Further, the system 100 may be configured to provide communication for transmitting and receiving signals between the monitoring device 102 and the analysis system 104. The analysis system 104 may comprise a network base module 124, a device network memory 126, a create transfer function module 128 and an execute transfer function module 130.

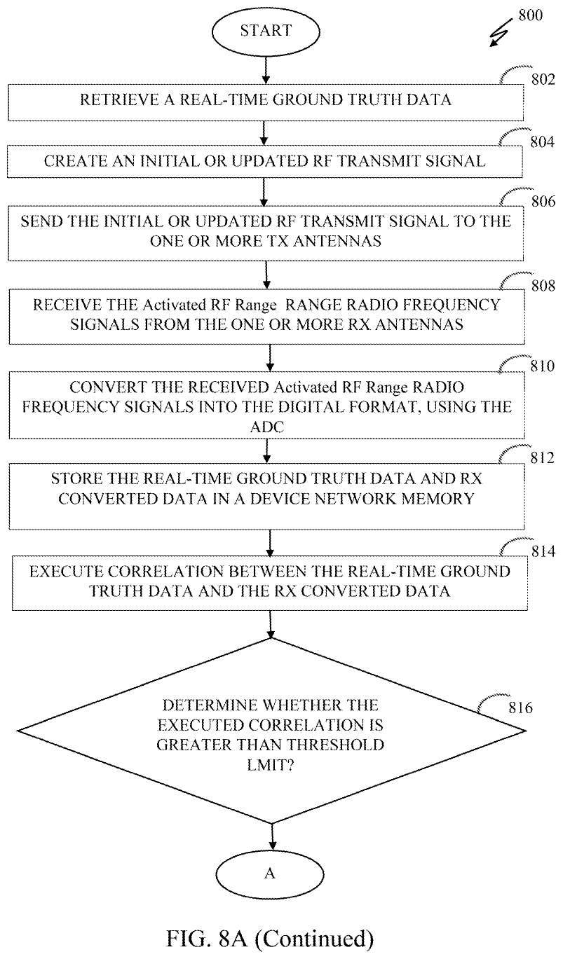

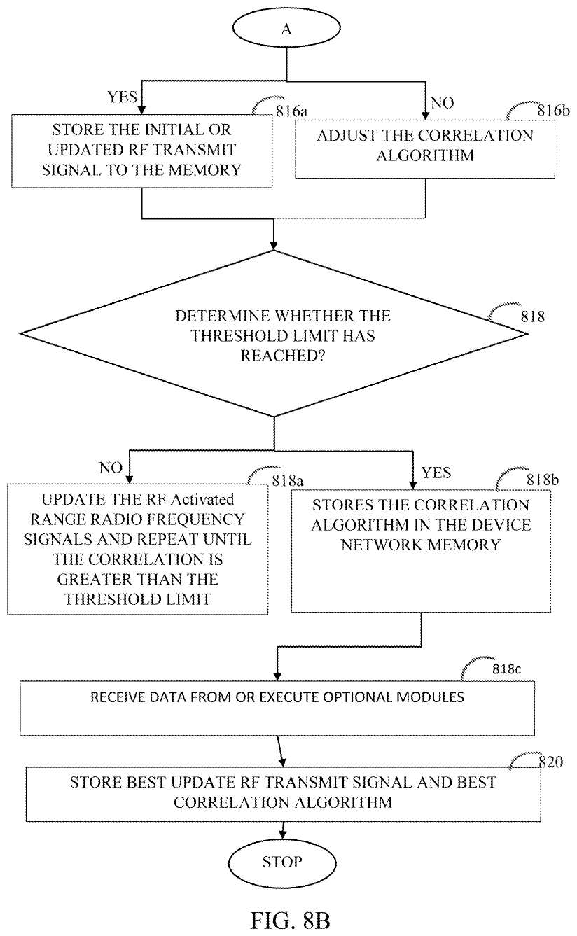

The analysis system 104 may be configured to receive polling data from the device base module 120 using the communication module 116. The polling data of the device base module 120 may be transmitted to the network base module 124. Further, the analysis system 104 comprises the device network memory 126 configured to store the polling data received from the monitoring device 102. In one embodiment, the device network memory 126 may be configured to store the filtered RF signal data received from the one or more RX antennas 108 of the monitoring device 102. Examples of implementation of the device network memory 126 may include, but are not limited to, Cloud storage, Cloud server, Random Access Memory (RAM), Read Only Memory (ROM), and/or a Secure Digital (SD) card. Further, the device network memory 126 may be configured to store a correlation algorithm generated by the create transfer function module 128. The create transfer function module 128 will be described in conjunction with FIG. 8. The correlation algorithm will be described later in conjunction with the create transfer function module 128. Further, the system 100 may be configured to facilitate the operation of the create transfer function module 128 and the execute transfer function module 130 to perform different operations for generating a suitable frequency signal for monitoring health parameters, specifically blood glucose level.

Further, the create transfer function module 128 may be configured to provide real-time comparisons between the received RX antennas signals and real-time ground truth data. In this way, received RX antennas signals can be matched to ground truth data. The real-time ground truth data may correspond to actual data related to health parameters in the blood sample of the user. The ground truth data may be obtained using a conventional device that is accepted as being accurate, and the ground truth data stored in the memory 126. For example, the create transfer function module 128 retrieves that the real-time ground truth data related to the blood glucose level of Bob is 110 mg/dL corresponding to a radio signal of frequency 122 GHz (It should be noted that this is an example of any frequency band from the Activated RF Range). It should be noted that real time ground truth data can be related to combined multiple frequency bands, for example, a 500 MHz band, a 700 MHz band, a 10 GHz band and a 122 GHz band. In one embodiment, the real-time ground truth data may also correspond to a known value or standard parameter value to be obtained. For example, the known ground truth data related to the blood glucose level is 125 mg/dL for an average adult in the United States of America.

Further, the create transfer function module 128 may create an initial or updated RF transmit signal from the real-time ground truth data. Further, the create transfer function module 128 may send the initial or updated RF transmit signal to TX antennas 106. Further, a signal resulting from transmitting the initial or updated RF signal may be received from the one or more RX antennas 108. The received signal resulting from transmitting the initial or updated RF signal may be converted to a digital signal using the ADC 110, as described earlier. Further, the create transfer function module 128 may be configured to store the real-time ground truth data and the converted initial or updated RF signal in the device network memory 126. Further, the create transfer function module 128 may be configured to execute the correlation algorithm between the real-time ground truth data and the converted initial or updated RF signal. Further, the create transfer function module 128 may be configured to determine whether the correlation is greater than or equal to a threshold limit. The threshold limit may correspond to a correlated value or coefficient between a derived value and a known value. In one embodiment, the threshold limit is less than 1. In another embodiment, the threshold limit is greater than or equal to 0.9. In one case, the create transfer function module 128 may determine that the correlation is greater than the threshold limit. The create transfer function module 128 may store the initial or updated RF transmit signal. In another case, the create transfer function module 128 may determine that the correlation is less than the threshold limit. The create transfer function module 128 may adjust the correlation algorithm in this case. In one embodiment, the create transfer function module 128 may adjust the correlation algorithm using auto-correlation, cross-correlation, positive, negative, and no correlation techniques.

In one embodiment, the create transfer function module 128 may determine whether the correlation reaches the threshold limit after adjusting the correlation. In one case, the create transfer function module 128 may determine that the correlation reaches the threshold limit. In this case, the system 100 is configured to store the correlation algorithm in the device network memory 126. In another case, the create function transfer module 128 may determine that the correlation algorithm does not reach the threshold limit. In this case, the create function transfer module 128 may update the initial or updated RF transmit signal and repeat until the correlation is greater than or equal to the threshold limit. Successively, the create transfer function module 128 is configured to store the best updated RF transmit signal and best correlation algorithm.

Further, the network base module 124 may be configured to transmit data to the cloud network 122 from the device base module 120. Further, the network base module 124 may store the received data in the device memory network 126. Successively, the network base module 124 may be configured to run the execute transfer function module 130. The network base module 124 may determine whether notification data exceeds the threshold limit. In one embodiment, the notification data may include information related to the best updated RF transmit signal. In this case, the network base module 124 may send a notification to the monitoring device 102.

In one embodiment, the analysis system 104 may comprise the execute transfer function module 130. The execute transfer function module 130 is configured to read the received data from the device network memory 126. The execute transfer function module 130 is configured to execute the best correlation algorithm on the signal resulting from transmitting the updated RF transmit signal. Further, the execute transfer function module 130 may be configured to determine whether the threshold limit is acceptable. In this case, the execute transfer function module 130 may send a notification signal to the monitoring device 102 via the network base module 124.

FIG. 2 illustrates a posterior view 200 of a hand 202 of the user with an approximate location of a cephalic vein 204 and a basilic vein 206 overlaid/superimposed, according to an embodiment.

In one embodiment, wearable electronics like smartwatches and health and fitness trackers may often be worn on the wrist, like conventional wristwatches or rubber bands. It has been shown that the wrist's anatomy is significant for measuring glucose levels with millimeter-range radio waves. The cephalic vein 204 and basilic vein 206 may be seen superimposed on the back of a right hand or hand 202 in FIG. 2. The left-side measurement of a wrist's depth yields a standard range of 40-60 mm (based on a wrist circumference in the range of 140-190 mm). The basilic vein 206 may be roughly located in subcutaneous tissue under the skin.

It can be noted that the thickness of human skin in a wrist area is around 1-4 mm, and the thickness of the subcutaneous tissue may vary from 1-34 mm, although these thicknesses may vary based on many factors. It can be noted that the hand 202 includes both capillaries having a diameter in the range of 5-10 microns, and the cephalic vein 204 and the basilic vein 206 having a diameter range of 1-4 mm. The capillaries, the cephalic vein 204, and the basilic vein 206 may be approximately 1-9 mm below the skin of the hand 202. In one embodiment, the Activated RF Range radio frequency signals may be particularly employed in pinpointing the position of a blood vessel like the basilic vein and thereby monitoring the blood glucose level.

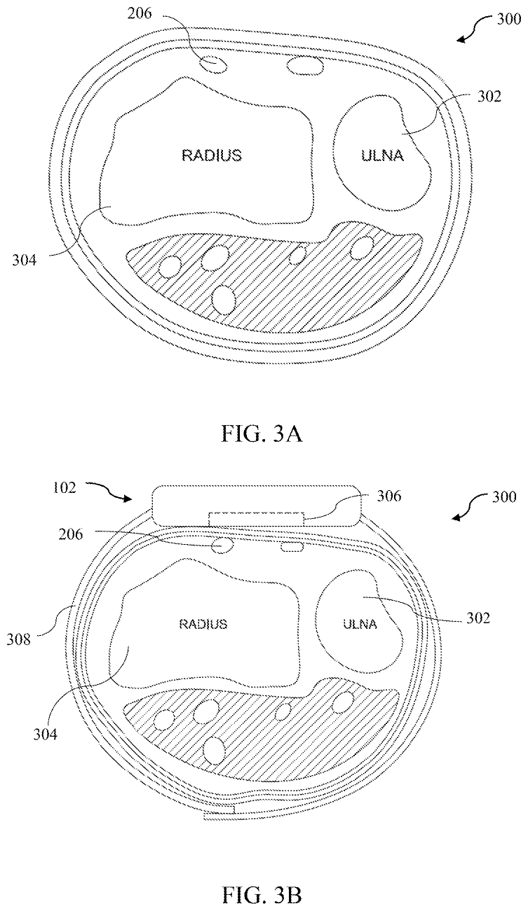

FIGS. 3A-3B illustrate a cross-sectional view of a wrist 300 with ulna and radius bones 302, 304 and the basilic vein 206, according to an embodiment.

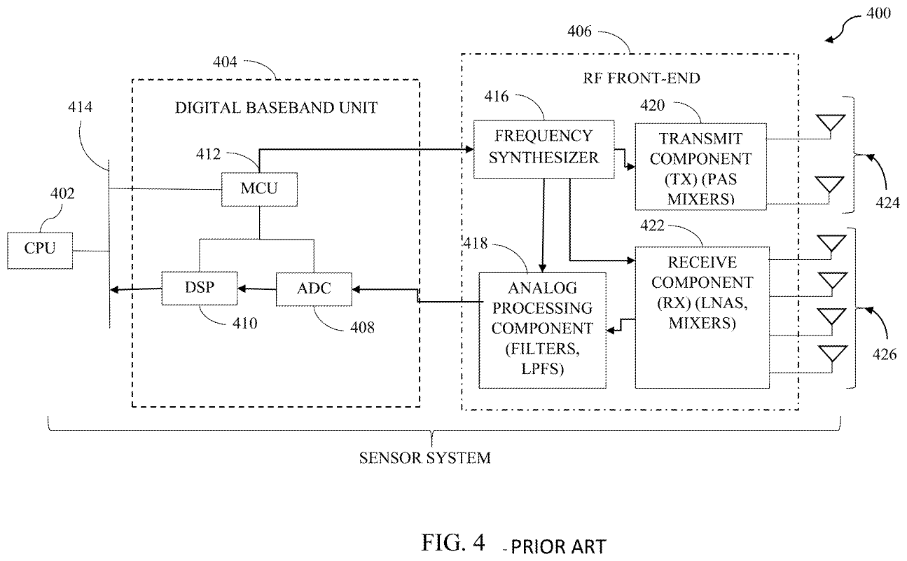

The wrist 300 may be provided with the monitoring device 102, as shown in FIG. 3B. In one example embodiment, the location of the monitoring device 102 relative to the wrist 300 and relative to the basilic vein 206 of the wrist 300 is depicted. The location of the monitoring device 102 relative to the anatomy of the wrist 300, including the radius bone 304, the ulna bone 302, and the basilic vein 206, is an important consideration in monitoring blood glucose levels using Activated RF Range radio frequency signals. Further, a dashed line block (shown by 306) represents an approximate location of a sensor system (not shown) on the monitoring device 102. The sensor system is described in conjunction with FIG. 4. Further, the monitoring device 102 may be provided with a strap 308 to tightly hold the monitoring device 102 around the wrist 300 in a secured position. It can be noted that the strap 308 may be provided with multiple fastening means (not shown) to adjust the monitoring device 102 around the wrist 300. The monitoring device 102 may be configured to transmit the Activated RF Range radio frequency signals towards the basilic vein 206 and receive the Activated RF Range radio frequency signals responded from blood components inside the basilic vein 206. It can be noted that a large quantity of Activated RF Range radio frequency signals imparted underneath the skin of the wrist 300 may be responded from the radius bone 304 and/or the ulna bone 302 in the wrist 300 as well as from some dense tissue, such as tendons and ligaments, that are located between the skin and the bones at a posterior of the wrist 300.

FIG. 4 illustrates a functional block diagram of a sensor system 400 of the monitoring device 102 utilizing the Activated RF Range radio frequency signals to monitor the blood glucose level in the user, according to an embodiment.

The sensor system 400 may comprise a central processing unit (CPU) 402, a digital baseband unit 404, and a radio frequency (RF) front end 406. Further, the digital baseband unit 404 may comprise an analog-to-digital converter (ADC) 408, a digital signal processor (DSP) 410, and a microcontroller unit (MCU) 412. In one embodiment, the digital baseband unit 404 may include some other configurations, including some other combination of elements. The digital baseband unit 404 may be connected to the CPU 402 using bus connectors 414.

Further, the RF front-end 406 may comprise a frequency synthesizer 416, an analog processing component 418, a transmit (TX) component 420, and a receive (RX) component 422. Further, the TX component 420 may include PAS elements. The PAS elements correspond to power, amplifiers, and mixers. The RX component 422 may include LNAS elements. The LNAS elements correspond to low noise amplifiers (LNAs), variable gain amplifiers (VGAs), and mixers. The frequency synthesizer 416 may include elements to generate electrical signals at frequencies used by the TX component 420 and the RX components 422. In one embodiment, the frequency synthesizer 416 may include elements such as a crystal oscillator, a phase-locked loop (PLL), a frequency multiplier, and a combination thereof. The analog processing component 418 may include elements such as mixers and filters. In one embodiment, the filters may include low-pass filters (LPFs). In one embodiment, the frequency synthesizer 416, the analog processing component 418, the TX component 420, and the RX component 422 of the RF front end 406 may be implemented in hardware as electronic circuits that are fabricated on the same semiconductor substrate.

Further, the TX component 420 may comprise at least two TX antennas 424, and the RX component 422 may comprise at least four RX antennas 426. In one embodiment, the sensor system 400 may be provided with multiple TX antennas and RX antennas in a ratio of 1:2.

Further, at least two TX antennas 424 and the at least four RX antennas 426 may be configured to transmit and receive activated RF range radio frequency signals. In one embodiment, the sensor system 400, including the CPU 402, the digital baseband unit 404, and the RF front end 406 of the monitoring device 102, may be integrated into various configurations according to the size and shape of the monitoring device 102. For example, some configurations of components of the monitoring device 102 is fabricated on a semiconductor substrate and/or included in a packaged IC device or a combination of packaged IC devices. In one embodiment, the monitoring device 102 is designed to transmit and receive millimeter radio frequency signals at a pre-defined frequency. In one embodiment, the pre-defined frequency ranges between 122-126 GHz for Activated RF Range radio frequency signals.

FIG. 5 illustrates a circuitry arrangement 500 of the one or more TX antennas 106 and the one or more RX antennas 108 of the sensor system 400 on a substrate 502 of the monitoring device 102, according to an embodiment. This is just one example of an arrangement of TX and RX antennas. For instance an arrangement may be just a single TX and RX antenna array, or two parallel TX and RX antenna arrays. Antenna arrays are designed to help insure an overlap to a vein.

The circuitry arrangement 500 may be fabricated on the substrate 502 with the one or more TX antennas 106 and the one or more RX antennas 108. Further, the circuitry arrangement 500 may be coupled to an input/output (I/O) interface 504. Further, the substrate 502 may have an outer footprint 506 and an inner footprint 508. The outer footprint 506 may correspond to an IC device of the monitoring device 102, and the inner footprint 508 may be a semiconductor substrate with circuits for the circuitry arrangement 500 and the one or more TX antennas 106 and the one or more RX antennas 108. The circuits are fabricated into the semiconductor substrate to conduct and process electrical signals transmitted by the one or more TX antennas 106 and received by the one or more RX antennas 108.

In one embodiment, the circuitry arrangement 500 has a footprint that is compatible to fitting on a wrist In one embodiment, the one or more TX antennas 106 and the one or more RX antennas 108 are designed specifically for Activated RF Range-range radio frequency signals.

In one embodiment, the one or more TX antennas 106 and the one or more RX antennas 108 are depicted as square boxes of approximately 1 mm×1 mm and are linked on the same planar surface of the substrate 502 to the circuitry arrangement 500.

For example, the one or more TX antennas 106 and the one or more RX antennas 108 are attached on the top surface of the circuitry arrangement 500 using a ceramic package material directly above the substrate 502 with conductive vias that electrically connect a conductive pad (not shown) of the substrate 502 to a transmission line of the one or more TX antennas 106 and the one or more RX antennas 108. In another embodiment, the one or more TX antennas 106 and the one or more RX antennas 108 may not have square arrangements. It can be noted that the square boxes correspond to an approximate footprint of the one or more TX antennas 106 and the one or more RX antennas 108. In one exemplary embodiment, the one or more TX antennas 106 and the one or more RX antennas 108 are microstrip patch antennas with dimensional functionality equivalent to the function of wavelength of the Activated RF Range radio frequency signals. In another exemplary embodiment, the one or more TX antennas 106 and the one or more RX antennas 108 correspond to antennas such as dipole antennas.

In one embodiment, some of the one or more TX antennas 106 and some of the one or more RX antennas 108 are positioned to alternate with one another. For example, in this embodiment we have three sets of one transmit 106 for each respective two receive antennas 108 arranged around the ring.

In one exemplary embodiment, each of the one or more TX antennas 106 includes channel-specific circuits (not shown) such as amplifiers, and each of the one or more RX antennas 108 includes channel-specific circuits (not shown) such as the mixers, the filters, and the LNAS, as described earlier. In another exemplary embodiment, the circuitry arrangement 500 includes a voltage control oscillator (VCO), a local oscillator (LO), frequency synthesizers, divider(s), mixers, ADCs, buffers, digital logic, DSPs, CPUs, and/or some combination thereof that may be utilized in conjunction with the channel-specific TX and RX antennas. Further, the one or more TX antennas 106 and the one or more RX antennas 108 may include an electrical interface (not shown) between a circuit on the substrate 502 and a corresponding antenna. In one embodiment, the electrical interface may be a conductive pad.

In one embodiment, the one or more TX antennas 106 and the one or more RX antennas 108 may be attached to the top surface of the substrate. It can be noted that the top surface may have a thickness of less than 0.5 mm. The one or more TX antennas 106 and the one or more RX antennas 108 may be connected to the electrical interface of each respective transmit/receive component separated by a fraction of a millimeter. Further, the substrate 502 may be integrated with a via perpendicular to the plane of the circuitry arrangement 500 and connected to the electrical interface of each respective transmit/receive component. In one embodiment, multiple vias may be used when an antenna of the one or more TX antennas 106 or the one or more RX antennas 108 is provided with more than one transmission line. Such a collocated configuration of the substrate 502 enables a desired distribution of the one or more TX antennas 106 and the one or more RX antennas 108 to be maintained while effectively managing conductor losses in the sensor system 400. In one embodiment, the one or more RX antennas 108 may form a phased antenna array for health monitoring applications. It is desirable to have as much spatial separation as possible between the one or more RX antennas 108 to improve overall signal quality by obtaining unique signals from each of the one or more RX antennas 108.

FIG. 6 illustrates the circuitry arrangement 500 of the one or more TX antennas 106 and the one or more RX antennas 108 of the sensor system 400 on the substrate 502 of the monitoring device 102 overlaid on the hand 202 of the user, according to an embodiment.

The circuitry arrangement 500, as explained in FIG. 5, is fabricated on the substrate 502 of the monitoring device 102 and overlaid on the wrist 300. The circuitry arrangement 500 may be oriented with respect to the basilic vein 206 and the cephalic vein 204,

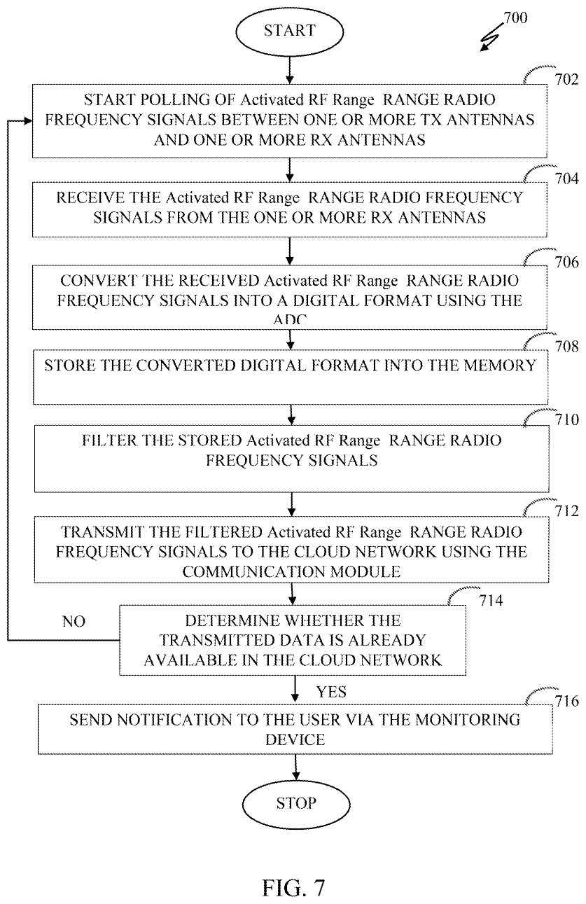

FIG. 7 illustrates a flowchart of a method 700 executed on the device base module 120, according to an embodiment.

At first, the device base module 120 may be configured to start polling the Activated RF Range radio frequency signals between the one or more TX antennas 106 and the one or more RX antennas, at step 702. In one embodiment, the device base module 120 may be configured to read and process instructions stored in the memory 112 using the processor 114. For example, the device base module 120 sends Activated RF Range radio signals of frequency range 100-126 GHz to a TX antenna and stores the Activated RF Range radio signals into the memory 112. The TX antenna sends the Activated RF Range radio signals underneath a patient's skin.

Further, the device base module 120 may receive the responses from the Activated RF Range radio frequency signals from the one or more RX antennas 108 at step 704. For example, an RX antenna receives a responded Activated RF Range radio signal of frequency range 100-110 GHz from the blood in the patient's blood vessels.

Successively, the device base module 120 may be configured to convert the received Activated RF Range radio frequency signals into a digital format using the ADC 110 at step 706. For example, the received Activated RF Range radio signal of frequency range 100-110 GHz is converted into a 10-bit data signal. Successively, the device base module 120 may be configured to store the converted digital format signal into the memory 112 at step 708.

Further, the device base module 120 may be configured to filter the stored Activated RF Range radio frequency signals at step 710. The device base module 120 may be configured in one embodiment to filter each stored Activated RF Range radio frequency signal using a low pass filter. For example, the device base module 120 filters the stored data.

Successively, the device base module 120 may be configured to transmit the filtered Activated RF Range radio frequency signals to the cloud network 122 using the communication module 116 at step 712. For example, the device base module 120 transmits data related to the transmission of an Activated RF Range frequency range 122-126 GHz to the cloud network 122.

Further, the device base module 120 may be configured to determine whether the transmitted data is already sent and available in the cloud network 122 at step 714. In one case, the device base module 120 determines that the transmitted data is not already present in the cloud network 122. In this case, the device base module 120 may be redirected back to step 702 to poll the Activated RF Range radio frequency signals between the one or more TX antennas 106 and the one or more RX antennas 108. For example, the device base module 120 determines that the transmitted Activated RF Range radio signal of frequency range 122-126 GHz is not present in the cloud network 122, and corresponding to the transmitted signal, there is no data related to the blood glucose level of the patient.

In another case, the device base module 120 determines that transmitted data is already present in the cloud network 122. For example, the device base module 120 reads cloud notification of the patient's blood glucose level as 110 mg/dL corresponding to a Activated RF Range radio signal of frequency range 122-126 GHz. In this case, the device base module 120 may proceed to step 716 to notify the user via the monitoring device 102.

FIGS. 8A-B illustrate a flowchart of a method 800 of the create the transfer function module 128, according to an embodiment.

At first, referring to FIG. 8A, the create transfer function module 128 is created using real-time ground truth data at step 802 and comparing that to signals in the Activated RF Range. In one embodiment, the real-time ground truth data corresponds to actual data related to health parameters in the blood sample of the user. For example, the create transfer function module 128 retrieves that the real-time ground truth data related to the blood glucose level of Bob is 110 mg/dL and it is found it corresponds to a radio signal of frequency 122 GHz. It should be noted that it could be found that the real time ground truth data corresponds to multiple frequency bands of the Activated RF Range response.

Further, the create transfer function module 128 may be configured to create an initial or updated RF transmit signal at step 804. In one embodiment, the initial or updated RF transmit signal may correspond to the Activated RF Range radio frequency signals transmitted from the one or more TX antennas 106 continuously in a loop. The method iterates in a “loop” to find the best transfer function correlation algorithm. Each loop may have a first signal, referred to as the initial RF transmit signal. After this first signal gets evaluated every other signal is referred to as the updated signal. Each of these updated signals are received from the RX antennas 108 in response to the TX antennas 106. In FIG. 8A the process starts. At step 802 the method retrieves a real-time ground truth data. At this point there is a glucose reading from the user. The user has the wireless RF wrist device worn, so that as transmitted signals are sent into the users wrist, the transmitted signals will be at the same time form the ground truth glucose reading on the patient.

In step 804 the method creates an initial or updated RF transmit signal. This means that a first RF signal is chosen from the Activated RF Range. If this step 804 was done on another loop, the signal would be the next or updated signal. The process is working to find the best RF signals that can be correlated to the ground truth data. Next step 806 sends the initial or updated rf transmit signal to the one or more TX antennas. Next in step 808 the method receives the Activated RF Range radio frequency signals from the one or more RX antennas 108. Next in step 810 the method converts the received radio frequency signals into the digital format, using the ADC. The ADC converts the continuous voltage or current levels of the analog signals into discrete digital values, allowing for digital signal processing and analysis. In step 812, the method stores the real-time ground truth data and RX converted data in a device network memory. Next, in step 814 the method executes correlations between the real-time ground truth data and the RX converted data. In order to correlate the real time RX received signals to ground truth data, the method evaluates all the received signals from the RX antennas to find which signals are the highest correlation to the ground truth data. In step 816, the method determines whether the executed correlation has results that is greater than a threshold. In this step it is determined if the given RX received signal matches more closely to the ground truth data than other RX received signals. A threshold is set that determines whether the match is not matched, closely matched or very closely matched by using the results of the correlation of the specific RX received signal to all the other RX received signals.

Successively, the create transfer function module 128 may be configured to receive the Activated RF Range radio frequency signals from the one or more RX antennas 108 at step 808. In one embodiment, the one or more RX antennas 108 may receive responded signals with respect to the Activated RF Range radio frequency signals transmitted into the user.

Further, the create transfer function module 128 may be configured to convert the received Activated RF Range radio frequency signals into the digital format, using the ADC 110, at step 810. In one embodiment, the signal received from the one or more RX antennas 108 may be converted into a suitable processable format to extract information related to a specific component in the blood sample. For example, the create transfer function module 128 using the ADC 110 converts the 750 MHZ into 10-bit data.

Successively, the create transfer function module 128 may be configured to store the real-time ground truth data, and RX converted data in the device network memory 126, at step 812. For example, the create transfer function module 128 stores the real-time ground truth data related to the blood glucose level of Bob as 110 mg/dL corresponding to the radio signal of frequency 122 GHz.

Further, the create transfer function module 128 may be configured to execute the correlation between the real-time ground truth data and the RX converted data at step 814. In one embodiment, the correlation between the real-time ground truth data and the RX-converted data is executed to determine whether the RX-converted data corresponds to the real-time ground truth data. For example, the create transfer function module 128 executes the correlation between the real-time ground truth data related to the blood glucose level of Bob as 110 mg/dL corresponding to the radio signal of frequency 122 GHz.

Successively, the create transfer function module 128 may be configured to determine whether the executed correlation is greater than the threshold limit at step 816. In one embodiment, the threshold limit may be a correlation coefficient greater than or equal to 0.9. In one case, referring to FIG. 8B, if the correlation is greater than the threshold limit, then the create transfer function module 128 stores the initial or updated RF and transmits a signal to the memory 112 at step 816a. For example, the create transfer function module 128 determines that the correlation is 0.97, which is greater than the threshold limit of 0.9. Thereafter, the create transfer function module 128 stores a frequency range 120-126 GHz.

In another case, the create transfer function module 128 may determine that all the RX received signals has a correlation that is not greater than the threshold limit at step 816. For example, the create transfer function module 128 determines that the best correlation is 0.79, which is less than the threshold limit of 0.9. In this case, the create transfer function module 128 may adjust the correlation algorithm at step 816b. In one embodiment, the create transfer function module 128 employs the auto-correlation technique to increase the correlation from 0.79 to 0.91. In this way, the method not only seeks the best match of the received signals to the ground truth data, it also seeks the best correlation method to use.

Successively, the create transfer function module 128 may determine whether the threshold limit has been reached at step 818. In one case, the create transfer function module 128 may determine that the threshold limit is not reached. For example, the create transfer function module 128 determines that the threshold limit is increased from 0.79 to 0.85, which is less than the threshold limit of 0.9. In this case, the create transfer function module 128 may update the Activated RF Range radio frequency signals and repeat until the correlation exceeds the threshold limit at step 818a. In another case, the create transfer function module 128 may determine that the threshold limit is reached. For example, the create transfer function module 128 determines that the threshold limit is increased to 0.9 from 0.79, which is equal to the threshold limit. In this case, the create transfer function module 128 stores the correlation algorithm in the device network memory 126 at step 818b. The create transfer function module 128 may optionally, at step 818c, receive data from, or execute the movement module 132 to determine if movement of the monitoring device 102 exceeds a threshold, above which, the RF signal data is unreliable. The create transfer function module 128 may optionally, at step 818c, receive data from, or execute the body temperature module 134 to determine if the user's body temperature or the environmental temperature around the monitoring device 102 exceeds a threshold, above which, the RF signal data is unreliable. The create transfer function module 128 may optionally, at step 818c, receive data from, or execute the body position module 136 to determine if the position of the monitoring device 102 relative to the user exceeds a threshold, above which, the RF signal data is unreliable. The create transfer function module 128 may optionally, at step 818c, receive data from, or execute the ECG module 138 to determine if the user's cardiac data exceeds a threshold, above which, the RF signal data is unreliable. The create transfer function module 128 may optionally, at step 818c, receive data from, or execute the circadian rhythm module 140 to determine if the user's sleep data exceeds a threshold, above which, the RF signal data is unreliable. The create transfer function module 128 may optionally, at step 818c, receive data from, or execute the received noise module 142 to determine if the background noise around the monitoring device 102 exceeds a threshold, above which, the RF signal data is unreliable.

Further, the create transfer function module 128 may store the best update RF transmit signal and best correlation algorithm at step 820. In one embodiment, the create transfer function module 128 may update and adjust the correlation algorithm multiple times during execution and correlation between the real-time ground truth data and the RX converted data. For example, the create transfer function module 128 stores a frequency range 140-155 GHz as the best updated RF transmit signal corresponding to the threshold coefficient of 0.97, which is greater than the threshold limit of 0.9.

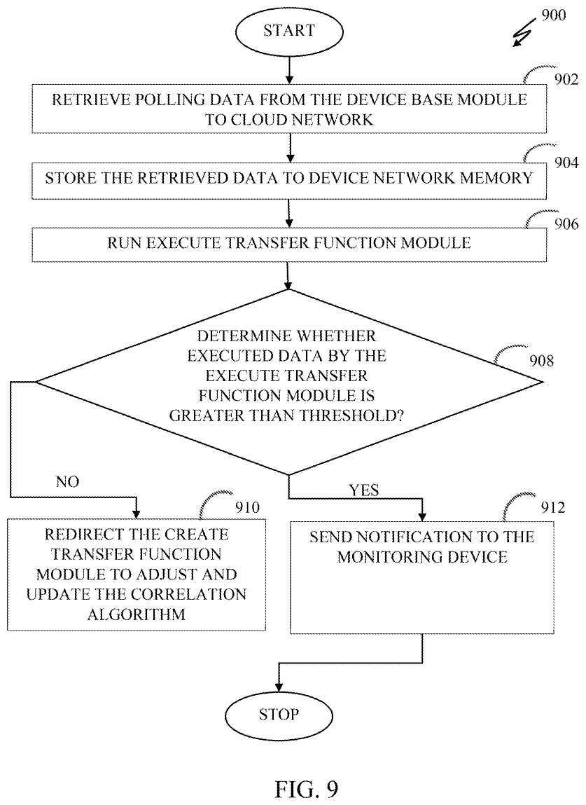

FIG. 9 illustrates a flowchart of a method 900 performed by the network base module 124, according to an embodiment.

At first, the network base module 124 may be configured to retrieve polling data from the device base module 120 at step 902. In one embodiment, the polling of the Activated RF Range radio frequency signals between the one or more TX antennas 106 and the one or more RX antennas 108, as mentioned in FIG. 7, is sent or retrieved to the network base module 124. For example, the network base module 124 retrieves a frequency range 100-110 GHz converted into a 10-bit data signal from the device base module 120.

Successively, the network base module 124 may be configured to store the retrieved data to device network memory 126 at step 904. For example, the network base module 124 stores a frequency range 100-110 GHz converted into a 10-bit data signal to the device network memory 126.

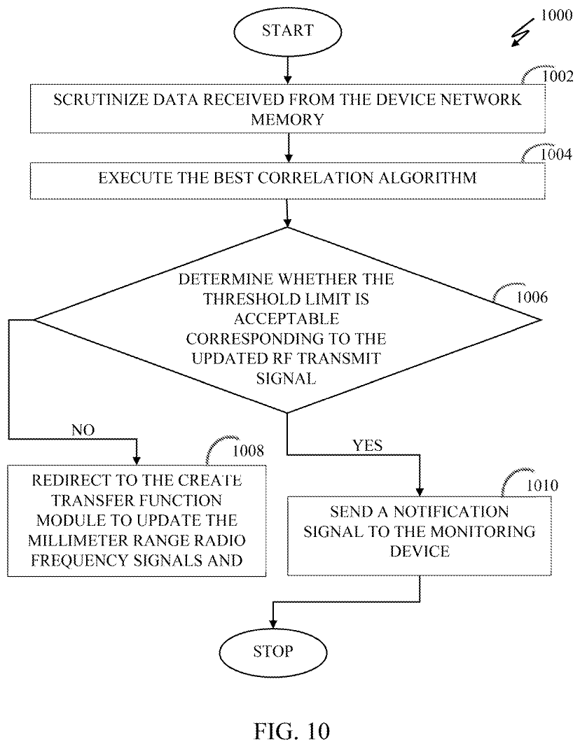

Further, the network base module 124 may be configured to run the execute transfer function module 130 at step 906. In one embodiment, the network base module 124 may run the stored best updated RF transmit signal and the best correlation algorithm created by the create transfer function module 128, as shown in FIGS. 8A-B. The execute transfer function module 130 is described in conjunction with FIG. 10.

Successively, the network base module 124 may be configured to determine whether executed data by the execute transfer function module 130 is greater than threshold limit at step 908. In one embodiment, the network base module 124 may receive notification data once the execute transfer function module 130 is operated. In one case, the network base module 124 may determine that the executed data is less than the threshold limit. For example, the network base module 124 determines that the executed data is 0.88, which is less than the threshold limit of 0.90. In this case, the network base module 124 may redirect the create transfer function module 128 to adjust and update the correlation algorithm at step 910. In another case, the network base module 124 may determine that the executed data exceeds the threshold limit. For example, the network base module 124 determines that the executed data is 0.98, which is greater than the threshold limit of 0.90. In this case, the network base module 124 may send a notification to the monitoring device 102 at step 912. For example, the network base module 124 sends a notification to the monitoring device 102 that the frequency range 140-155 GHz is the best updated RF transmit signal corresponding to the threshold coefficient of 0.98 is greater than the threshold limit of 0.9.

FIG. 10 illustrates a flowchart of a method 1000 performed by the execute transfer function module 130, according to an embodiment.

At first, the execute transfer function module 130 may be configured to scrutinize data received from the device network memory 126 at step 1002. The execute transfer function module 130 may be provided with the data to execute the operation for determining the health parameter of the user. In one embodiment, the execute transfer function module 130 may determine the user's blood glucose level from the best updated RF transmit signal and the best correlation algorithm stored in the memory 112 and the device network memory 126. For example, the execute transfer function module 130 reads that the best updated RF transmit signal stored in the device network memory.

Successively, the execute transfer function module 130 may be configured to execute the best correlation algorithm at step 1004. For example, the execute transfer function module 130 runs a frequency range 140-155 GHz. Further, the execute transfer function module 130 may be configured to determine whether the threshold limit is acceptable corresponding to the updated RF transmit signal at step 1006. In one case, the execute transfer function module 130 may determine that the threshold limit is unacceptable. For example, the execute transfer function module 130 determines that the threshold limit is 0.87, which is less than an acceptable limit of 0.90. In this case, the execute transfer function module 130 may redirect to the create transfer function module 128 to update the Activated RF Range radio frequency signals and repeat until the threshold limit is acceptable at step 1008. In another case, the execute transfer function module 130 may determine that the threshold limit is acceptable. For example, the execute transfer function module 130 determines that the threshold limit is 0.92, slightly greater than the acceptable limit of 0.90. In this case, the execute transfer function module sends a notification signal to the monitoring device 102 at step 1010. For example, the execute transfer function module 130 sends that the 65-millimeter radio signal of frequency range 140-155 GHz is acceptable to determine the patient's blood glucose level.

US 2020/0187813 is incorporated by reference herein in its entirety.

1. A health parameter monitoring system, comprising:
a monitoring device configured for communicative coupling to an analysis system, including:
one or more transmit antennas configured to transmit radio-frequency (RF) analyte detection signals into a user over the space below a skin surface and one or more receive antennas configured to detect RF analyte signals that result from the RF analyte detection signals transmitted into the user;
an analog-to-digital converter connected to the one or more receive antennas and receiving the RF analyte signals detected by the one or more receive antennas;
a substrate on which the analog-to-digital converter and the one or more transmit antennas and the one or more receive antennas are fabricated, the transmit antennas and receive antennas forming one or more antenna arrays; and
a sensor comprising at least one of: a movement sensor, a body temperature sensor, a body position sensor, and an electrocardiogram sensor, that senses user data during transmission of the RF analyte detection signals by the one or more transmit antennas and during detection of the RF analyte signals by the one or more receive antennas.

2. The health parameter monitoring system of claim 1, further comprising a background noise sensor that senses background noise data during detection of the RF analyte signals by the one or more receive antennas.

3. The health parameter monitoring system of claim 1, wherein the RF analyte detection signals include information related to a blood pressure related analyte.

4. The health parameter monitoring system of claim 1, wherein the monitoring device is wearable.

5. The health parameter monitoring system of claim 4, wherein the monitoring device is one of: a cell phone; a smartwatch; a tracker; a wearable monitor; a wristband; and a personal blood monitoring device.

6. The health parameter monitoring system of claim 1, wherein the substrate is fabricated with at least two transmit antennas, and at least four receive antennas.

7. The health parameter monitoring system of claim 1, wherein the substrate has an outer footprint and an inner footprint, wherein the outer footprint corresponds to an integrated circuit device and the inner footprint is a semiconductor substrate with a circuit arrangement.

8. The health parameter monitoring system of claim 1, further including a memory connected to the analog-to-digital converter in which digital signals are stored.

9. The health parameter monitoring system of claim 1, wherein the sensor provides elevation information related to hand and heart positions of a user.

10. The health parameter monitoring system of claim 1 further including a circadian rhythm sensor that senses one or more of actigraphy, wrist temperature, light exposure, and heart rate during transmission of the RF analyte detection signals and during detection of the RF analyte signals.

11. The health parameter monitoring system of claim 1, wherein the body position sensor is one of a group of sensors including: an accelerometer, a gyroscope and an inertial movement sensor.

12. The health parameter monitoring system of claim 1, further including a communications module providing communication to an analysis system via a cloud network.

13. The health parameter monitoring system of claim 12, wherein the communications module sends communications via wireless communication protocols.

14. A health parameter monitoring method, comprising:
detecting an analyte in a user by transmitting radio-frequency (RF) analyte detection signals into the user over the space below a skin surface from one or more transmit antennas and detecting, using one or more receive antennas, RF analyte signals that result from the RF analyte detection signals transmitted into the user;
converting the detected RF analyte signals from analog signals to digital signals using an analog-to-digital converter connected to the one or more receive antennas;
sensing parameter data of the user using one of a movement sensor, a body temperature sensor, or a body position sensor, during transmission of the RF analyte detection signals and during detection of the RF analyte signals; and
storing the digital signals and parameter data in a memory connected to the analog-to-digital converter.

15. The health parameter monitoring method of claim 14, further comparing the sensed parameter data of the user to a threshold stored in a memory.

16. The health parameter monitoring method of claim 15, further including modifying the digital signals by filtering using a low band pass filter.

17. The health parameter monitoring method of claim 16, further including transmitting the digital signals after filtering to a cloud network using a communication module.
